(19) United States
(12) Patent Application Publication
(10) Pub. No.: US 2022/0008192 A1
(43) Pub. Date: Jan. 13, 2022

(54) DEVICE FOR SUPPORT AND SHAPE OF SOFT TISSUE

(71) Applicant: Vanderbilt University, Nashville, TN (US)

(72) Inventor: Galen Perdikis, Nashville, TN (US)

(21) Appl. No.: 17/373,411

(22) Filed: Jul. 12, 2021

Related U.S. Application Data
(60) Provisional application No. 63/050,595, filed on Jul. 10, 2020.

(51) Int. Cl. A61F 2/12

(52) U.S. Cl. CPC A61F 2/12; A61F 2220/0075; A61F 2210/0004

(57) ABSTRACT

A device for use in surgical procedures, such as breast reconstruction procedures. The device holds and shapes soft tissue during healing and comprises materials that naturally dissolve within the human body.

### CROSS-REFERENCE TO RELATED APPLICATIONS

This application is a non-provisional of and claims the benefit of U.S. Provisional Patent Application No. 63/050,595, filed on Jul. 10, 2020, the contents of which are incorporated herein by reference.

### FIELD OF THE DISCLOSURE

The present disclosure relates to a tissue holder and shaper for use in surgical procedures, such as breast reconstruction procedures.

### SUMMARY

In one embodiment, the disclosure provides a device for tissue reconstruction. The device comprises a body formed from a material that is dissolvable and resorbable, the body including a base and a wall extending from the base, a recess formed in the base, the recess configured to receive patient tissue, wherein the body is configured to be secured to a chest wall of the patient.

In another embodiment, the disclosure provides a device for tissue reconstruction. The device comprises a hollow body formed from a material that is dissolvable, the body defining a height, a width, and a depth, and a plurality of openings extending through the body, wherein the body is configured to receive and support tissue and be temporarily secured to a structure within a patient's body.

In addition, other aspects of the disclosure will become apparent by consideration of the detailed description and accompanying drawings.

### BRIEF DESCRIPTION OF THE DRAWINGS

FIG. 1 is a bottom perspective view of a tissue holder and shaper according to a first embodiment of the invention.

FIG. 2 is a side view of the tissue holder and shaper of FIG. 1.

FIG. 3 is a front view of the tissue holder and shaper of FIG. 1.

### DETAILED DESCRIPTION

Before any aspects of the disclosure are explained in detail, it is to be understood that the disclosure is not limited in its application to the details of construction and the arrangement of components set forth in the following description or illustrated in the following drawings. The disclosure is capable of supporting other aspects and being practiced or being carried out in various ways. Also, it is to be understood that the phraseology and terminology used herein is for the purpose of description and should not be regarded as limiting.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example, “about 1” may also mean from 0.5 to 1.4.

A “subject” or “patient” may be human or non-human and may include, for example, animal strains or species used as “model systems” for research purposes, such as a mouse model. Likewise, patient may include either adults or juveniles (e.g., children). Moreover, patient may mean any living organism, preferably a mammal (e.g., human or non-human) that may benefit from application or use of the disclosed device.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Patients who require breast reconstruction surgery generally require tissue to reconstruct the breast. Sometimes a flap of tissue is taken from the abdomen of the patient. In particular, sometimes a “TRAM flap” is used to reconstruct the breast. The TRAM flap incorporates fatty tissue, skin, and an abdominal muscle from the patient to construct a natural implant. Other times a “DIEP flap” is used to reconstruct the breast. The DIEP flap incorporates only fatty tissue and skin to construct the natural implant. Other times an artificial implant can be used for breast reconstruction.

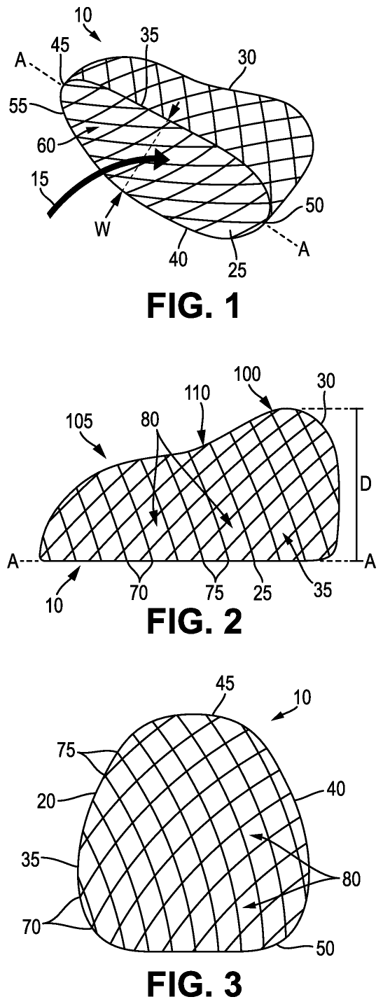

FIGS. 1-3 illustrate a device 10 for use in breast reconstruction surgeries to support, hold, and shape soft tissue 15 (e.g., a flap). The device 10 includes a hollow body 20 that has a longitudinal axis A, a base 25, and a wall 30 extending from the base 25. The base 25 has a first side 35, a second side 40 opposite the first side 35, a first end 45, a second end 50 opposite the first end 45, and an opening 55 that provides access to a recess 60. The opening 55 and the recess 60 are positioned between the first side 35 and the second side 40.

In the illustrated embodiment, the opening 55 and the recess 60 are offset (e.g., not centered) along the longitudinal axis A. The flap 15 is positionable within the recess 60 through the opening 55. The base 25 may have a height H and a width W. The height H is measured between opposite ends 45, 50 of the base 25 along the longitudinal axis A and the width W is measured between opposite sides 35, 40, as shown in FIG. 1. In the illustrated embodiment, the width W is variable along the longitudinal axis A. In one construction, the height H can range from about 10 cm to about 17 cm, and the maximum width can range from about 10 cm to about 18 cm. For example, in one embodiment, the height H can be about 12 cm and the maximum width can be about 16 cm. The body 20 is trimmable such that the body 20 (e.g., the height H and width W) can be any suitable size for the patient.

As shown in FIGS. 1 and 2, the wall 30 may be formed from a plurality of first strands 70 and a plurality of second strands 75. Each of the plurality of first and second strands 70, 75 extend from the first side 35 of the base 25 to the second side 40 of the base 25. Each of the plurality of first strands 70 extend generally in a first direction from the first side 35 and each of the plurality of second strands 75 extend generally in a second, different direction from the first side 35. In the illustrated embodiment, each of the first and second strands 70, 75 extend from and are coupled to the base 25 at a non-parallel and non-perpendicular angle. Accordingly, each of the plurality of first strands 70 intersects one or more of the plurality of second strands 75 and vice versa. The plurality of first and second strands 70, 75 can intermesh to form a lattice or mesh structure. In the illustrated embodiment, the first and second strands 70, 75 are integrally formed with one another as a unitary structure. Together the plurality of first and second strands 70, 75 define a lattice or mesh structure having a plurality of openings 80. As shown, each of the openings 80 is positioned between two adjacent first strands 70 and two adjacent second strands 75. The wall 30, and therefore each of the first and second strands 70, 75, can have a thickness ranging from about 1 mm to about 3 mm. In one construction, one or more of the plurality of openings 80 may be sized about 3 mm by about 3 mm. Preferably, the openings 80 are as large as possible while still retaining the ability to support the flap 15, as discussed below. In some constructions, the openings 80 can be at least about 10 mm by about 10 mm. In some constructions, the maximum dimension (e.g., width) of the openings 80 may be up to about 1 cm. In some constructions, the maximum dimension of the openings 80 may be up to about 2 cm. In other constructions, the plurality of first strands 70 and the plurality of second strands 75 can be positioned relative to the base 25 and to one another in other ways to create the lattice or mesh structure. For example, the plurality of first strands 70 may extend generally perpendicular to the longitudinal axis A, while the plurality of second strands 75 may extend generally parallel to the longitudinal axis A.

With respect to FIG. 2, in the illustrated embodiment, the wall 30 defines an anatomically-shaped device for supporting and shaping soft tissue. That is, the wall 30 defines a projection profile including a first concave portion 100 (e.g., a first projection), a second concave portion 105 (e.g., a second projection), and a convex portion 110 between the first and second concave portions 100, 105. The first concave portion 100, the second concave portion 105, and the convex portion 110 are aligned along the longitudinal axis A such that, when viewed from the side, the wall 30 defines an undulating surface. The concavities (e.g., radii of curvature) of the first concave portion 100, the second concave portion 105, and the convex portion 105 are different. Accordingly, a depth of the projection profile D measured between a distal edge of the wall and the base 25 is variable along the height H of the body 20 and along the longitudinal axis A. The depth D may measure from about 3 cm to about 10 cm. The wall 30 may have other suitable configurations or projection profiles in other embodiments. For example, the wall 30 can define a single uniformly arcuate portion such that the device 10 is substantially round.

The body 20 of the device 10 is configured to shape and support the flap 15 positioned in the recess 60. Accordingly, the body 20 is, in one construction, formed of a material that is sufficiently rigid to support the flap 15 and sufficiently pliable or flexible to mimic the anatomical structure (e.g., the breast) being reconstructed. The material is also dissolvable and resorbable. In some embodiments, the material is dissolvable over a period of time that can range between about three weeks to about three months. In some embodiments, the material is dissolvable within about three months to about six months. In some embodiments, the material is dissolvable within about six months to about one year. In some embodiments, the material may be vicryl, polydioxanone (PDS), poliglecaprone (Monocryl), poly-4-hydroxybutyrate (galaflex), tubularized Sodium hyaluronate/carboxymethylcellulose (seprafilm), and/or Tubularized polylactide (Surgiwrap). In some embodiments, the material may be catgut, polyglycolic acid, polylactic acid, polydioxone, caprolactone or a combination of these. Other suitable materials may also or additionally be used. Additionally, the material may be configured to stretch by up to 20% depending on the application. For example, the material used for soft/pliable tissue replacement, such as for breast reconstruction, is configured to have greater stretch than the material used for more rigid tissue replacement, such as joint or spine reconstruction.

In some embodiments, the device 10 can include a drain (not shown). For example, the device 10 can include one or more dissolvable tubes having one or more holes and are comprised of polyglactin, polydioxanone, or combinations thereof. The tubes, like the device 10, are dissolvable. Additionally, the device 10 can also include an antibacterial agent (e.g., Irrisept or the like) that is slowly released over time (e.g., one day to a week). In some embodiments, the antibacterial agent may be integral with the material of the body 20. In some embodiments, the antibacterial agent may be applied (as a spray or a gel) to the body 20.

For use in breast reconstruction surgery, the flap 15 is positioned within the body 20 of the device 10. Specifically, the flap 15 is inserted into the body 20 and positioned in the recess 60. The body 20 is then appropriately positioned relative and secured (e.g., by sutures or the like) to the chest wall of the patient. The device 10 temporarily shapes the newly reconstructed breast and immediately gives it projection, while the flap 15 adheres to the chest wall and surrounding tissue on its own over time. The device 10 supports the soft tissue and gradually dissolves leaving only the flap 15 behind. The device 10 of the present disclosure is novel in view of other similar devices, which are sheet-like or flat and do not have a pliable three dimensional structure and therefore cannot give the reconstructed breast immediate shape and projection.

The device 10 shown in FIGS. 1-3 is configured to be used in breast reconstruction surgery. In other embodiments, the device 10 can be configured for use in other types of surgical procedures requiring temporary support for an implant facial reconstruction plates (e.g., reconstruction bars or jigs for mandible) and/or reconstruction with fibula, plates, and/or bone.

Although the disclosure has been described in detail with reference to certain preferred aspects, variations and modifications exist within the scope and spirit of one or more independent aspects of the disclosure as described. Various features and advantages of the disclosure are set forth in the following claims.

What is claimed is:

1. A device for tissue reconstruction, the device comprising:
a body formed from a material that is dissolvable and resorbable, the body including a base and a wall extending from the base;
a recess formed in the base, the recess configured to receive patient tissue;
wherein the body is configured to be secured to a chest wall of the patient.

2. The device of claim 1, wherein the wall includes a lattice structure.

3. The device of claim 2, wherein the lattice structure defines openings that are greater than 3 mm by 3 mm.

4. The device of claim 2, wherein the lattice structure includes a plurality of first strands and a plurality of second strands, wherein the plurality of first strands and the plurality of second strands are coupled to the base at a non-parallel and non-perpendicular angle.

5. The device of claim 4, wherein the plurality of first strands and the plurality of second strands have a thickness ranging from about 1 mm to about 3 mm.

6. The device of claim 4, wherein the body defines a longitudinal axis, and wherein the plurality of first strands extend perpendicular to the longitudinal axis, and wherein the plurality of second strands extend parallel to the longitudinal axis.

7. The device of claim 1, wherein the wall includes a first side and a second side opposite the first side, and wherein the first side includes a concave portion.

8. The device of claim 4, wherein the first side includes a first concave portion and a second concave portion having a different concavity than the first concave portion.

9. The device of claim 1, wherein the body has a depth measured from the base to a distal edge of the wall, the depth ranging from 3 mm to 10 mm.

10. A device for tissue reconstruction, the device comprising:
a hollow body formed from a material that is dissolvable, the body defining a height, a width, and a depth; and
a plurality of openings extending through the body, and
wherein the body is configured to receive and support tissue and be temporarily secured to a structure within a patient's body.

11. The device of claim 10, wherein the depth of the body is variable along the height of the body.

12. The device of claim 10, wherein the body includes a plurality of first strands and a plurality of second strands, wherein the plurality of first strands and the plurality of second strands are coupled to a base of the body at a non-parallel and non-perpendicular angle.

13. The device of claim 12, wherein the plurality of first strands and the plurality of second strands have a thickness ranging from about 1 mm to about 3 mm.

14. The device of claim 12, wherein the body defines a longitudinal axis, and wherein the plurality of first strands extend perpendicular to the longitudinal axis, and wherein the plurality of second strands extend parallel to the longitudinal axis.
